(19) United States
(12) Patent Application Publication
(10) Pub. No.: US 2009/0035369 A1
(43) Pub. Date: Feb. 5, 2009

(54) Composition and Methods for Treating and Preventing Age-Related Macular Degeneration

(76) Inventor: Yoram Sela, Raanana (IL)

Correspondence Address: NIXON & VANDERHYE, PC, 901 NORTH GLEBE ROAD, 11TH FLOOR, ARLINGTON, VA, 22203, US

(21) Appl. No.: 12/224,376

(22) PCT Filed: Feb. 25, 2007
(86) PCT No.: PCT/IL2007/000247

§ 371 Date: Aug. 26, 2008

(30) Foreign Application Priority Data
Feb. 27, 2006 (IL) 173971

(51) Int. Cl. A61K 31/404; A61K 33/30; A61K 33/34; A61K 9/14; A61K 9/28; A61K 9/48; A61K 9/50; A61P 27/02

(52) U.S. Cl. 424/456; 514/415; 424/641; 424/630; 424/490; 424/474

(57) ABSTRACT

The present invention relates to methods and compositions effective in preventing, attenuating, inhibiting the progression of, and treating eye diseases, e.g., age-related macular degeneration (AMD). The compositions comprise, as an active ingredient, an effective amount of 3,3′-diindolylmethane (DIM) and/or its precursor indole-3-carbinol (I3C). The compositions can further comprise additional agents such as carotenoids and other phytochemicals, which produce a synergistic effect in preventing, attenuating, inhibiting the progression of, and treating AMD and other eye diseases such as glaucoma, cataracts and diabetic retinopathy (DR). The compositions are particularly effective in treating and preventing wet AMD, and in preventing or inhibiting the progression of dry AMD (non-neovascular) to wet AMD (neovascular).

### FIELD OF THE INVENTION

The present invention relates to the field of ophthalmology, in particular to pharmaceutical compositions comprising 3,3′-diindolylmethane (DIM) and/or its precursor indole-3-carbinol (I3C), and their use in treating age-related macular degeneration (AMD), in particular wet AMD.

### BACKGROUND OF THE INVENTION

### Eye Pathologies

Eye diseases, including age-related macular degeneration (AMD), cataracts, diabetic retinopathy and glaucoma, are a leading cause of blindness affecting millions of people worldwide. As the global population ages, the number of individuals affected is expected to increase substantially.

Macular degeneration, or age-related macular degeneration (AMD), affects the central part of the retina and is the leading cause of blindness in people over age 65 in the United States. AMD affects 13 million people and causes impairment in about 1.2 million. The prevalence of AMD increases with age from 16.8% in patients 55-64 to 25.6% in patients 65-74 and up to 42% in patients over 75. AMD is caused by hardening of the arteries that nourish the retina, which deprives the retinal tissue of oxygen and, leading to vision deterioration.

There are two types of AMD: dry (nonneovascular) and wet (neovascular). Dry AMD accounts for about 90 percent of all cases and is sometimes referred to as atrophic, nonexudative, or drusenoid macular degeneration. In dry AMD, deposits called drusen accumulate in the retinal pigment epithelium (RPE) tissue beneath the macula. These deposits are thought to interfere with the function of photoreceptors in the macula, causing progressive degeneration of these cells. Vision loss from dry AMD occurs very gradually over the course of many years.

Dry AMD often progresses into wet AMD. In wet AMD, abnormal blood vessels grow beneath the macula, and leak blood and fluid into the macula, causing injury to the retina and promoting scarring of the fovea (central macula). The macula has the highest concentration of photoreceptors facilitating central vision and permitting high-resolution visual acuity. The damage caused by the leakage and fibrovascular scarring in wet AMD leads to profound loss of central vision and severe loss of visual acuity (usually 20/200 or worse). Wet AMD is classified into several categories by the pattern of leakage on Flurescein Angiography: A) Classic: a well demarcated area of hyperfluorescence in the early frames of the fluorescein angiogram with increased fluorescence caused by pooling of the dye in the late phases of the study; B) Occult: early frames show poorly demarcated areas of hyperfluorescence, with persistent and increased staining in the late phases of the study. More often associated with subretinal blood, fluid, and exudates; C) Mixed: a mixture of classic and occult neovascular patterns on the fluorescein angiogram; and D) Recurrent: classic, occult or mixed type neovascular patterns on the fluorescein angiogram with a previous history of leakage or treatment (most commonly laser photocoagulation).

Without treatment, most patients with occult, classic, mixed, and recurrent type wet macular degeneration will end up with poor vision. In general, patients with a “classic” component lose vision more quickly. People with wet AMD have several limitations, including inability to read, inability to recognize faces or drive, and the disease often leads to blindness. The onset of severe visual changes in wet AMD can occur suddenly. More than 400,000 Americans are currently affected by this form of the disease, and the incidence is rising rapidly with the aging of the population. If wet AMD is diagnosed early, laser surgery can prevent extensive central vision loss by destroying the leaky blood vessels. No non-surgical treatments, however, are currently available.

3,3′-diindolylmethane

3,3′-diindolylmethane (DIM) and its precursor indole-3-carbinol (I3C) are plant indoles found in cruciferous vegetables including cabbage, broccoli, Brussels sprouts, and cauliflower. I3C is the natural precursor to DIM, which is formed from a condensation reaction of two I3C molecules. DIM has been shown to promote beneficial estrogen metabolism into its 2-hydroxy metabolites in both men and women. The benefits of this phytochemical include cardio and brain protective activities, cancer prevention, and benefits for perimenopausal women in premenstrual syndrome (PMS), endometriosis, and cervical dysplasia. Women on estrogen replacement (HRT) also benefit from supplementation, as well as men with estrogen-related conditions, including prostate hypertrophy. I3C and DIM have also been shown to have multiple anti-cancer effects both in in-vivo and in-vitro models. For example, I3C and DIM were shown to decrease the incidence of 7,12-dimethylbenz(a)anthracene (DMBA)-induced mammary tumors in female Sprague-Dawley rats, and to inhibit benzo(a)pyrene (BP)-induced neoplasia of the forestomach of mice (Wattenberg, L. W., 1978, Cancer Res. 38: 1410-1413; Grubbs et al. 1995, Anticancer Res. 15: 709-716; Stowsand et al. 1988, Cancer Lett., 39: 199-207; Chen e al. 1998; Carcinogenesis, 19: 1631-1639).

Pure DIM is insoluble and poorly absorbed by the human body. Thus, to obtain the benefits of DIM, very large quantities of DIM would need to be consumed. To overcome this problem, absorbable forms of pure DIM have been developed as dietary supplements that use special absorption-enhancing formulas. U.S. Pat. No. 6,086,915 to Zeligs et al. discloses spray dried hydrophobic chemopreventative compositions comprising phytochemicals such as DIM, a process for making such compositions and a method of using such compositions to adjust steroid metabolism in mammals. The hydrophobic dietary compositions are purported to enhance absorptivity when taken orally as a chemopreventative agent.

Several reports have linked DIM to angiogenesis or vascular endothelial growth factor (VEGF). McCarty et al. reported that DIM's cancer growth inhibition effect may be largely attributable to an angiostatic action (McCarty et al. 2005, Integr Cancer Ther, 4:301-14). Chang et al. found that DIM can inhibit VEGF-induced cell proliferation and DNA synthesis in human umbilical vascular endothelial cells (Chang et al., 2005, Carcinogenesis, in print). Aggrawal et al. reported that DIM inhibits the activation of various transcription factors, including nuclear ractor-E2-related factor 2 (Nrf2), and that the pharmacological activity of the precursor I3C is mediated through, inter alia, its ability to inhibit invasion and angiogenesis (Aggrawal, B. B. et al. 2005, Cell Cycle, 4: 1201-15). The antiangiogenic properties of DIM were also reported by Chang et al. who found that DIM inhibits up to 76% of neovascularization in a rodent model (Chang, X., et al. 2005, Carcinogenesis, 26: 771-8). It should be mentioned, however, that DIM is not a pure anti-angiogenic or anti-VEGF agent, i.e., it does not inhibit the VEGF-receptor, but rather inhibits downstream activities of the receptor.

### Dietary Supplements and Antioxidants:

Dietary supplements have been shown to be effective in reducing the risk of and in treating certain eye diseases. The Age-Related Eye Disease Study (AREDS), a major clinical trial performed to evaluate the effect of high oral doses of antioxidants and zinc on the progression of AMD and cataract, showed that the consumption of high levels of antioxidants and zinc reduce the risk of developing advanced AMD by about 25 percent. The AREDS composition comprises vitamin A, vitamin C, vitamin E and the minerals zinc and copper. Intake of this composition was shown to be associated with side effects, including genitourinary problems, anemia, skin yellowing and an increased risk of lung cancer in smokers.

Another study, the Lutein Antioxidant Supplement Trial (LAST), evaluated the effects of orally administered lutein and lutein combined with antioxidants on atrophic (dry) AMD. Both treatments significantly improved some measures of visual function, including glare recovery, contrast sensitivity, and visual acuity. The long-term safety of dietary lutein supplementation has not yet been determined.

Carotenoids are naturally occurring pigments found in certain plants and algae. For example, the red color of tomatoes and yellow color of corn derive from carotenoids. Mammals are incapable of synthesizing carotenoids in situ and must obtain them through their diet. High dietary intake of carotenoids has been shown to be associated with reduced incidence of certain types of cancers, cardiovascular disease and hypertension. A dermal photoprotective effect is also associated with a high dietary intake of carotenoids.

Epidemiological studies have shown that dietary anti-oxidants such as carotenoids delay the progression of eye disease, including the progression of AMD. Epidemiological studies have established that a low serum concentration of carotenoids, especially lycopene, the main tomato carotenoid, is associated with risk of AMD. Other carotenoids, including lutein and zeaxanthin, have been shown to have a protective role in the development of cataract and macular degeneration (Mares-Perlman J A et al., 2002 J Nutr. Mar; 132(3): 518S-524S.

Lycopene

Lycopene is the major carotenoid present in the diet and provides the familiar red color of tomato products. More than 80% of dietary intake of lycopene is derived from tomato sources, such as ketchup; tomato juice, spaghetti sauce, tomato soup and pizza sauce. Lycopene can be prepared synthetically, or can be obtained as a natural tomato extract. For example, lycopene is obtainable under the name Lyc-O-Mato® (LycoRed, Israel) as an all-natural antioxidant formula containing tomato lycopene, tocopherols; beta-carotene, phytoene, phytofluene, tomato oil, phospholipids, and other important bioactive phytochemicals naturally occurring in tomato oleoresin.

Lycopene has been shown to provide eye protection and endogenous protection of UV induced skin damage when administered orally. Several studies have shown an anti-cataract effect of dietary lycopene in animals (Pollack A, et al. 1996-1997, Metab Pediatr Syst Ophthalmol. 19-20: 31-6.). Lycopene prevents galactose-induced morphological changes in cultured human lens cells, suggesting its use as an anti-cataract agent (Mohanty, I, et al. 2002, British J Nutrition, 88: 347-354). Without wishing to be bound to theory, it is believed that lycopene has a protective effect on lutein and zeaxanthin.

Lutein and Zeaxanthin

Lutein and its stereoisomer zeaxanthin belong to the xanthophyll family of carotenoids and are the only two known carotenoid specifically accumulated from plasma and deposited in the lens and macula lutea. The macular pigment comprises lutein and zeaxanthin, and functions as a filter to protect the light-sensitive photoreceptor cells that are also known to scavenge reactive oxygen species (ROS). Dietary intake of these carotenoids results in increased macular pigment and improved vision in patients with AMD and other ocular diseases (reviewed in Alves-Rodrigues, A. and Shao, A. 2004 Toxicology Letters, 150: 57-83).

Green leafy vegetables are the best dietary source of lutein, with spinach, kale and parsley providing high levels. Purified crystalline lutein has been classified generally recognized as safe (GRAS) and can be added to food and beverages. Pure lutein may also be isolated from certain plants, as described, for example, in U.S. Pat. No. 5,382,714 and U.S. Pat. No. 5,648,564.

Seddon et al. reported the first direct relationship between carotenoid intake and AMD risk. Lutein and zeaxanthin were most strongly associated with a decreased AMD risk. An oral dose of 6% wt/wt per day was shown to translate into a 57% lower risk of disease (Seddon J M et al. 1994, JAMA. 272(18):1413-20).

Carnosic Acid, Phytoene and Phytofluene

Carnosic acid is an antioxidant extracted from rosemary (Rosemarinus spp) and other herbs, which has been shown to inhibit LDL oxidation in a synergistic manner with lycopene. Phytoene and phytofluene are carotenoids found in tomatoes, and may be found in tomato oleoresin.

There remains an unmet need in the art compositions that are effective in preventing and treating eye diseases such as AMD, in particular wet AMD

### SUMMARY OF THE INVENTION

The present invention relates to methods and compositions effective in preventing, attenuating, inhibiting the progression of, and treating eye diseases, e.g., age-related macular degeneration (AMD). The compositions comprise, as an active ingredient, an effective amount of 3,3′-diindolylmethane (DIM) and/or its precursor indole-3-carbinol (I3C). The compositions can further comprise additional agents such as carotenoids and other phytochemicals, which produce a synergistic effect in preventing, attenuating, inhibiting the progression of, and treating AMD and other eye diseases such as glaucoma, cataracts and diabetic retinopathy (DR). The compositions are particularly effective in treating and preventing wet AMD, and in preventing or inhibiting the progression of dry AMD (non-neovascular) to wet AMID (neovascular).

The present invention thus provides, in one embodiment, a method of preventing, attenuating, inhibiting the progression of, or treating AMD, by administering to a subject in need thereof a composition comprising at least one agent selected from DIM and I3C; and a pharmaceutically acceptable carrier or excipient, in an amount effective to prevent, attenuate, inhibit the progression of or treat AMD, preferably wet AMD.

In another aspect, the present invention provide pharmaceutical compositions useful for preventing, attenuating, inhibiting the progression of, or treating AMD, preferably wet AMD, the compositions comprising at least one agent selected from DIM and I3C; and a pharmaceutically acceptable carrier or excipient.

In another aspect, the present invention relates to the use of DIM, its precursor I3C or a combination thereof, in the manufacture of a medicament for preventing, attenuating, inhibiting the progression of or treating AMD, preferably wet AMD.

In one embodiment, the AMD is wet AMD. In another embodiment, the composition is provided in an amount effective to prevent, inhibit or attenuate the progression of dry AMD to wet AMD.

In one embodiment, the composition comprises DIM. In another embodiment, the composition comprises I3C. In a further embodiment, the composition comprises a combination of DIM and I3C. The active ingredient or ingredients are provided in a collective amount effective to prevent, attenuate, inhibit the progression of, or treating AMD especially wet AMD. For example, for DIM, the composition preferably comprises from about 25 mg to about 500 mg DIM, more preferably from about 50 mg to about 150 mg DIM. For I3C, the composition preferably comprises from about 25 mg to about 500 mg I3C, more preferably from about 50 mg to about 150 mg I3C.

Certain carotenoids and other phytochemicals are known to be effective in reducing the risk of certain eye diseases and may even improve visual function when ingested in high concentrations. Thus, in one embodiment, the compositions of the invention can further comprise at least one carotenoid, preferably lycopene. Lycopene can by synthetically prepared, but is preferably provided in the composition as a natural compound. In some embodiments, lycopene is provided as an extract, example, as an extract of tomato oleoresin, such as Lyc-O-Mato®.

In some embodiments the compositions can further comprise one or more additional phytochemicals, such as zeaxanthin and its isomer lutein, and adjuvants such as phytoene, phytofluene and carnosic acid or derivatives thereof (e.g., carnosol, 6,7-dehydrocarnosic acid or 7-ketocarnosic acid). If provided, these phytochemical(s), independently of each other, can be separately added to the composition, or they can be present as part of a natural extract, e.g., a lycopene extract. In some embodiments the phytochemicals are synthetically prepared.

Additional agents, including minerals, vitamins, and other micronutrients can optionally be incorporated in the compositions of the present invention.

In some embodiments, the composition further comprises a source of zinc, which can be zinc oxide or a zinc salt. Any zinc salt that is compatible with the eye is acceptable. Examples of zinc salts include but are not limited to zinc chloride, zinc acetate, zinc gluconate, zinc carbonate, zinc sulfate, zinc borate, zinc nitrate and zinc silicate.

In certain embodiments a source of copper is provided in the present composition. In some embodiments cupric oxide is preferred.

Certain preferred vitamins include vitamin A, vitamin C and vitamin E. The vitamins can be provided as natural or synthetically produced compounds. Derivatives, including provitamins of the preferred vitamins are acceptable. In some preferred embodiments vitamin A is provided as provitamin A, specifically β-carotene. In some embodiments, combinations of the above vitamins are included in the composition. The addition of vitamin E, vitamin C and β-carotene is preferred.

In one embodiment, the composition comprises at least one agent selected from DIM and I3C, and further comprises lycopene, lutein, zeaxanthin, phytoene, phytofluene, carnosic acid; and a pharmaceutically acceptable carrier or excipient

In another embodiment, the composition comprises at least one agent selected from DIM and I3C, and further comprises lycopene, lutein, zeaxanthin, phytoene, phytofluene, carnosic acid, vitamin A, vitamin C, vitamin E, zinc and copper; and a pharmaceutically acceptable carrier or excipient

In one currently preferred embodiment, the composition comprises DIM, lycopene, phytoene, phytofluene, lute in, zeaxanthin, and carnosic acid.

In another currently preferred embodiment, the composition comprises DIM, lycopene, phytoene, phytofluene, lutein, zeaxanthin, carnosic acid, vitamin A, vitamin C, vitamin E, zinc and copper.

In another currently preferred embodiment, the composition further comprises tomato oleoresin.

In another currently preferred embodiment, the composition further comprises at least one solubility enhancer to enhance the solubility of DIM, I3C or the combination thereof.

The present invention relates, in one embodiment, to pharmaceutical compositions for oral use or for topical administration to the eye. In one embodiment the composition is formulated for oral use in a form selected from a tablet, caplet, capsule, microcapsule, pellet, pill, powder, syrup, gel, slurry, granule, suspension, dispersion, emulsion, liquid, solution, dragee, bead and beadlet and the like. A beadlet is a polysaccharide complex, in the shape of a bead, in which small droplets containing the active material are embedded. In certain preferred embodiments the composition is formulated as a soft gelatin capsule or as a hard gelatin capsule. In other embodiments the composition is formulated as a beadlet based on alginates, gelatin or other natural or synthetic polymers.

The present invention also relates, in another embodiment, to pharmaceutical compositions formulated for application to the eye. The composition can be formulated as a liquid, cream, paste, ointment, emulsion including submicron emulsion, gel, thermogel, or suspension.

The compositions can also be dispensed as dry formulation, for example as powder, granules, microcapsules or capsules, for reconstitution as a liquid, dispersion, emulsion or suspension.

In some embodiments the composition is provided in an aqueous or non-aqueous medium, and is preferably sterile (microbe-free).

To date, there are no non-surgical options to treat wet AMD. The present invention addresses the deficiency in the art, by providing therapeutically powerful compositions, useful for preventing and treating AMD, especially wet AMD.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE PRESENT INVENTION

The present invention relates to methods and compositions effective in preventing, attenuating, inhibiting the progression of, and treating eye diseases, e.g., age-related macular degeneration (AMD). The compositions comprise, as an active ingredient, an effective amount of 3,3′-diindolylmethane (DIM), and/or its precursor indole-3-carbinol (I3C). The compositions can further comprise additional agents such as carotenoids and other phytochemicals, which produce a synergistic effect in preventing, attenuating, inhibiting the progression of, and treating AMD and other eye diseases such as glaucoma, cataracts and diabetic retinopathy (DR). The compositions are particularly effective in treating and preventing wet AMD, and in preventing or inhibiting the progression of dry AMD (non-neovascular) to wet AMD (neovascular).

There are several known commercial formulations of DIM, which are marketed as supplements to enhance estrogen metabolism. For example, DIM is marketed as BioDIM®, an oral capsule dosage form comprising 75 mg DIM, phosphatidylcholine, vitamin E (as d-alpha tocopheryl succinate), microcrystalline cellulose and hydroxypropylmethyl cellulose. DIM is also marketed in an oral tablet dosage form containing 100 mg DIM, vitamin E, phosphatidyl choline, pepper fruit extract, dibasic calcium phosphate, stearic acid, modified cellulose gum, magnesium stearate and colloidal silicon dioxide. DIM-Plus™ is another commercial formulation containing BioResponse-DIM™, an enhanced bioavailability delivery system for DIM. The formulation is provided in an oral capsule dosage form, containing 100 mg BioResponse-DIM™ (enhanced bioavailability complex containing 25% DIM), starch, vitamin E, phosphatidylcholine, silica, and Protectamins® (spinach powder, cabbage powder, concentrated broccoli powder), cellulose, gelatin and chlorophyll.

There are also several known commercial formulations of I3C. For example, I3C is marketed as Vcap®, an oral capsule dosage form containing 200 mg I3C, LinumLife™ Complex (Flax Seed Lignan Extract Linum usitatissimum), cellulose, rice bran, magnesium stearate and silica. I3C is also marketed as Meta I3C™, an oral tablet dosage form containing 150 mg indole-3-carbinol and, Rosemary Leaf Extract (Rosmarinum officinalis).

Any of the above-mentioned or otherwise known commercial formulations, or any other formulations formulated for oral or ophthalmic delivery, can advantageously be used in the methods of the present invention.

According to one exemplary embodiment, DIM is provided in the present composition in a concentration range of about 1% w/w to about 75% w/w. A range of about 10% w/w to about 50% w/w is preferred, and a concentration range of about 35-40% w/w is more preferred.

According to another exemplary embodiment, I3C is provided in the present composition in a concentration range of about 1% w/w to about 75% w/w. A range of about 10% w/w to about 50% w/w is preferred, and a concentration range of about 35-40% w/w is more preferred.

According to another exemplary embodiment, a combination of DIM and I3C is provided in the present composition in a total concentration of both agents in the range of about 1% w/w to about 75% w/w. A range of about 10% w/w to about 50% w/w is preferred, and a concentration range of about 35-40% w/w is more preferred.

In one preferred embodiment, the compositions of the invention comprise DIM, I3C or a combination thereof as the sole active ingredients. In other embodiment, however, the compositions can further comprise additional agents such as carotenoids and other phytochemicals and their adjuvants.

### Carotenoids

Certain carotenoids and other phytochemicals are known to be effective in reducing the risk of certain eye diseases and may even improve visual function when ingested in high concentrations. Thus, in one currently preferred embodiment, the composition further comprises at least one carotenoid. Carotenoids useful in the compositions and methods of the present invention can be naturally occurring carotenoids found in, for example, tomato products (e.g., tomatoes, tomato sauce, ketchup and the like), fermentation, watermelon, guava, grapefruit, and other fruits and vegetables containing these carotenoids. The carotenoids can be obtained by extracting the carotenoid from a natural source, using extraction techniques known to a person of skill in the art, or they can be synthetically prepared using any total or semi-synthesis known to a person of skill in the art. Also contemplated are carotenoids derived from genetically modified organisms.

Examples of carotenoids include but are not limited to lycopene, α-carotene, β-carotene, zeta-carotene, α-cryptoxanthin, β-cryptoxanthin, phytoene, phytofluene, lutein, zeaxanthin, astaxantin, canthaxanthin, and combinations thereof.

A currently preferred carotenoid is lycopene. Lycopene can by synthetically prepared, but is preferably provided in the composition as a natural compound. In some embodiments, lycopene is provided as an extract, example, as an extract of tomato oleoresin, such as Lyc-O-Mato®.

According to one exemplary embodiment, lycopene is provided in the present composition in a concentration range of about 0.025% w/w to about 5% w/w. A range of about 0.25% w/w to about 2.5% w/w is preferred, and a concentration of about 1.5% w/w is more preferred.

In some embodiments, the compositions can further comprise one or more additional carotenoids, such as zeaxanthin and its isomer lutein. If provided, these carotenoids can, independently of each other, be separately added to the composition, or they can be present as part of a natural extract, e.g., a lycopene extract. In some embodiments, the phytochemicals are synthetically prepared.

In one exemplary embodiment, the compositions of the invention comprise lutein. In some embodiments, lutein is present in the composition in a concentration range of about 0.025% w/w to about 5% w/w. A range of about 0.25% w/w to about 2.5% w/w is preferred, and a concentration of about 2% w/w is more preferred. In some embodiments lutein is synthetically prepared. In other embodiments lutein is isolated and purified from a natural source.

In another exemplary embodiment, the compositions of the invention comprise zeaxanthin, a stereoisomer of lutein. In one exemplary embodiment, zeaxanthin is present in the composition in a concentration range of about 0.001% w/w to about 2% w/w. A range of about 0.025% w/w to about 1% w/w is preferred, and a concentration of about 0.5% w/w is more preferred. In some embodiments zeaxanthin is synthetically prepared. In other embodiments zeaxanthin is isolated and purified from a natural source.

In other embodiments, zeaxanthin is contained in the lutein fraction. In accordance with these embodiments, the total concentration of lutein and zeaxanthin is about 0.035% to about 7% w/w, preferably in the range of about 1.5% to about 2.5% w/w, more preferably about 2% to about 2.2% w/w total lutein and zeaxanthin.

Natural sources containing the carotenoids include various fruits and vegetables, as well as various animal products. Lycopene, phytoene and phytofluene are found in tomatoes. Beta carotene (provitamin A), is the carotenoid that gives carrots their orange color, and is converted to vitamin A in the walls of the small intestine (intestinal mucosa) in a reaction catalysed by the enzyme beta-carotene dioxygenase. Alpha carotene is also found in carrots and mixed vegetables. Beta-Cryptoxanthin, also known as cryptoxanthin, cryptoxanthol, and hydroxy-beta-carotene belongs to the xanthophylls class of carotenoids, and is also considered a pro-vitamin A since it can be converted to vitamin A in the human body. Beta-cryptoxanthin can be found in many vegetables and fruits, mainly in papaya, mango, peaches, oranges, tangerines, bell peppers, corn and watermelon. Beta-cryptoxanthin is also found in some yellow colored animal products such as egg yolk and butter. Astaxanthin is found in orange vegetables and in dark leafy greens. It can also be found in seafood such as salmon, trout, red seabream, shrimp, lobster and fish eggs. Canthaxanthin is known mainly as the natural pigment of the orange-yellow Chanterelle mushroom, but also occurs in various lower animals, some crustaceans, insects, fishes and birds. Spinach, kale, collard greens, romaine lettuce, leeks, peas, and egg yolks are good sources of lutein. Sources of zeaxanthin include corn, spinach, collards, oranges and other citrus products, lettuce, peas, beans, broccoli, celery, peaches and carrots.

A single carotenoid as well as combinations and mixtures thereof can be administered in the methods of the present invention. Accordingly, carotenoid mixtures of lycopene and phytoene; lycopene and phytofluene; and lycopene, phytoene, and phytofluene can be administered in the presently claimed methods and compositions. In a currently preferred embodiment, the carotenoid is lycopene. In another currently preferred embodiment, the carotenoid comprises a mixture of lycopene and phytoene. In another currently preferred embodiment, the carotenoid comprises a mixture of lycopene and phytofluene. In yet another currently preferred embodiment, the carotenoid comprises a mixture of lycopene, phytoene and phytofluene.

### Adjuvants

In some embodiments, the compositions can further comprise adjuvants such as phytoene, phytofluene and carnosic acid or derivatives thereof. If provided, these phytochemical(s) can, independently of each other, be separately added to the composition, or they can be present as part of a natural extract, e.g., a lycopene extract. In some embodiments the phytochemicals are synthetically prepared. In other embodiments the phytochemicals are purified from a natural source.

In one embodiment, phytoene and phytofluene are provided as adjuvants in the present compositions. In an exemplary embodiment, phytoene and phytofluene are present at about 10% of the concentration of lycopene. In another exemplary embodiment, each of phytoene and phytofluene are provided in a concentration range of about 0.0025% w/w to about 1.25% w/w. A range of about 0.025% w/w to about 0.25% w/w of each phytochemical is preferred, and a concentration of 0.15% w/w total of both phytoene and phytofluene, corresponding to about 10% of the lycopene content, is more preferred.

In other embodiments, carnosic acid or derivatives thereof, (e.g., carnosol, 6,7-dehydrocarnosic acid or 7-ketocarnosic acid) is also provided as an adjuvant in the present compositions. Carnosic acid or its derivatives is preferably provided in a concentration range of about 0.025% w/w to about 2.5% w/w. A range of about 0.25% w/w to about 2% w/w is preferred, and a concentration of 1% w/w is more preferred

### Supplements

Additional agents, including minerals, vitamins, and other micronutrients can optionally be incorporated in the compositions of the present invention.

In some embodiments the composition further comprises a source of zinc, which can be zinc oxide or a zinc salt. Any zinc salt that is compatible with the eye is acceptable. Examples of zinc salts include but are not limited to zinc chloride, zinc acetate, zinc gluconate, zinc carbonate, zinc sulfate, zinc borate, zinc nitrate and zinc silicate. In some embodiments a concentration of about 0.25% to about 15% w/w zinc is provided. In preferred embodiments a concentration of about 7.5% w/w zinc is provided; preferably in the form of zinc gluconate.

In certain embodiments a source of copper is provided in the present composition. In some embodiments cupric oxide is preferred. In some embodiments a copper is provided at a concentration of about 0.01% to about 5% w/w; preferably at a concentration of about 0.25% w/w.

Certain preferred vitamins include vitamin A, vitamin C and vitamin E. The vitamins can be provided as natural or synthetically produced compounds. Derivatives, including provitamins of the preferred vitamins are acceptable. In some preferred embodiments vitamin A is provided as provitamin A, specifically β-carotene. In some embodiments, combinations of the above vitamins are included in the composition. The addition of vitamin E, vitamin C and β-carotene is preferred. Vitamin E and vitamin C can each be included in the present composition at a concentration of about 0.25% to about 25% w/w. Preferable concentrations of vitamin E and vitamin C are about 12.5%. β-carotene can be included in the present composition at a concentration of about 0.025% to 2.5% w/w. Preferable concentrations of β-carotene are about 0.75% w/w.

Certain exemplary compositions will now be described. It is apparent to a person of skill in the art that the compositions described below are provided for purpose of illustration only, and do not limit the broad scope of the present invention.

In one exemplary embodiment, the composition comprises at least one agent selected from DIM and I3C, and further comprises lycopene, lutein, zeaxanthin, phytoene, phytofluene, carnosic acid, and a pharmaceutically acceptable carrier or excipient.

In another embodiment, the composition comprises at least one agent selected from DIM and I3C, and further comprise lycopene, lutein, zeaxanthin, phytoene, phytofluene, carnosic acid, vitamin A, vitamin C, vitamin E, zinc and copper; and a pharmaceutically acceptable carrier or excipient

In one currently preferred embodiment, the composition comprises DIM, lycopene, phytoene, phytofluene, lutein, zeaxanthin, and carnosic acid.

In another currently preferred embodiment, the composition comprises DIM, lycopene, phytoene, phytofluene, lutein, zeaxanthin, carnosic acid, vitamin A, vitamin C, vitamin E, zinc and copper.

In another currently preferred embodiment, the composition further comprises tomato oleoresin. The tomato oleoresin can be obtained from tomato or tomato products in accordance with methods well known to a person of skill in the art.

In one embodiment, the composition of the present invention comprises
from about 10% w/w to about 50% w/w DIM;
from about 0.025% w/w to about 5% w/w lutein;
from about 0.001% w/w to about 2% w/w zeaxanthin;
from about 0.025% w/w to about 5% w/w mg lycopene;
from about 0.0025% w/w to about 1.25% w/w mg phytoene;
from about 0.0025% w/w to about 1.25% w/w phytofluene;
from about 0.025% w/w to about 2.5% w/w carnosic acid or derivative thereof;
and a pharmaceutically acceptable carrier or excipient.

In a currently preferred embodiment, the composition of the present invention comprises; about 40% DIM, about 1.5% w/w lycopene; about 2% w/w total of lutein and zeaxanthin, about 0.15% w/w total of phytoene and phytofluene; about 1% w/w carnosic acid or a derivative thereof; and a pharmaceutically acceptable carrier or excipient.

In another embodiment, the composition of the present invention comprises
from about 10% w/w to about 50% w/w I3C
from about 0.025% w/w to about 5% w/w lutein;
from about 0.001% w/w to about 2% w/w zeaxanthin;
from about 0.025% w/w to about 5% w/w mg lycopene;
from about 0.0025% w/w to about 1.25% w/w mg phytoene;
from about 0.0025% w/w to about 1.25% w/w phytofluene;
from about 0.025% w/w to about 2.5% w/w carnosic acid or derivative thereof;
and a pharmaceutically acceptable carrier or excipient.

In a currently preferred embodiment, the composition of the present invention comprises; about 40% I3C, about 1.5% w/w lycopene; about 2% w/w total of lutein and zeaxanthin, about 0.15% w/w total of phytoene and phytofluene; about 1% w/w carnosic acid or a derivative thereof; and a pharmaceutically acceptable carrier or excipient.

In another embodiment, the composition of the present invention comprises
from about 10% w/w to about 50% w/w DIM;
from about 0.025% w/w to about 5% w/w lutein;
from about 0.001% w/w to about 2% w/w zeaxanthin;
from about 0.025% w/w to about 5% w/w mg lycopene;
from about 0.0025% w/w to about 1.25% w/w mg phytoene;
from about 0.0025% w/w to about 1.25% w/w phytofluene;
from about 0.025% w/w to about 2.5% w/w carnosic acid or derivative thereof;
from about 0.25% w/w to about 25% w/w vitamin E;
from about 0.25% w/w to about 25% w/w vitamin C;
from about 0.025% to about 2.5% w/w carotene;
from about 0.25% w/w to about 15% w/w mg zinc;
from about 0.01% w/w to about 5% w/w copper; and
a pharmaceutically acceptable carrier or excipient.

In a currently preferred embodiment, the composition comprises: about 40% DIM about 1.5% w/w lycopene; about 2% w/w total of lutein and zeaxanthin, about 0.15% w/w total of phytoene and phytofluene; about 1% w/w carnosic acid or a derivative thereof; about 12.5% w/w vitamin E; about 12.5% w/w vitamin C; about 0.75% w/w β-carotene; about 7.5% w/w zinc; about 0.25% w/w copper; and a pharmaceutically acceptable carrier or excipient.

In another embodiment, the composition of the present invention comprises
from about 10% w/w to about 50XX% w/w I3C;
from about 0.025% w/w to about 5% w/w lutein;
from about 0.001% w/w to about 2% w/w zeaxanthin;
from about 0.025% w/w to about 5% w/w mg lycopene;
from about 0.0025% w/w to about 1.25% w/w mg phytoene;
from about 0.0025% w/w to about 1.25% w/w phytofluene;
from about 0.025% w/w to about 2.5% w/w carnosic acid or derivative thereof;
from about 0.25% w/w to about 25% w/w vitamin E;
from about 0.25% w/w to about 25% w/w vitamin C;
from about 0.025% to about 2.5% w/w β-carotene;
from about 0.25% w/w to about 15% w/w mg zinc;
from about 0.01% w/w to about 5% w/w copper; and
a pharmaceutically acceptable carrier or excipient.

In a currently preferred embodiment, the composition comprises: about 40% I3C about 1.5% w/w lycopene; about 2% w/w total of lutein and zeaxanthin, about 0.15% w/w total of phytoene and phytofluene; about 1% w/w carnosic acid or a derivative thereof; about 12.5% w/w vitamin E; about 12.5% w/w vitamin C; about 0.75% w/w β-carotene; about 7.5% w/w zinc; about 0.25% w/w copper; and a pharmaceutically acceptable carrier or excipient.

### Therapeutic Use

As contemplated herein, the present invention provides powerful methods for preventing, attenuating, inhibiting the progression of, and treating eye diseases, e.g., age-related macular degeneration (AMD), especially wet AMD.

Thus, in one embodiment, the present invention provides a method of preventing AMD, by administering to a subject in need thereof a composition comprising DIM; and a pharmaceutically acceptable carrier or excipient, in an amount effective to prevent AMD, preferably wet AMD.

In another embodiment, the present invention provides a method of attenuating the progression of AMD, by administering to a subject in need thereof a composition comprising DIM; and a pharmaceutically acceptable carrier or excipient, in an amount effective to attenuate the progression of AMD, preferably wet AM.

In another embodiment, the present invention provides a method of inhibiting the progression of AMD, by administering to a subject in need thereof a composition comprising DIM; and a pharmaceutically acceptable carrier or excipient, in an amount effective to inhibit the progression of AMD, preferably wet AMD.

In another embodiment, the present invention provides a method of treating AMD, by administering to a subject in need thereof a composition comprising DIM; and a pharmaceutically acceptable carrier or excipient, in an amount effective to treat AMD.

In another embodiment, the present invention provides a method of preventing AMD, by administering to a subject in need thereof a composition comprising I3C; and a pharmaceutically acceptable carrier or excipient, in an amount effective to prevent AMD, preferably wet AMD.

In another embodiment, the present invention provides a method of attenuating the progression of AMD, by administering to a subject in need thereof a composition comprising I3C; and a pharmaceutically acceptable carrier or excipient, in an amount effective to attenuate the progression of AMD, preferably wet AMD.

In another embodiment, the present invention provides a method of inhibiting the progression of I3C, by administering to a subject in need thereof a composition comprising DIM; and a pharmaceutically acceptable carrier or excipient, in an amount effective to inhibit the progression of AMD, preferably wet AMD. in another embodiment, the present invention provides a method of treating AMD, by administering to a subject in need thereof a composition comprising I3C; and a pharmaceutically acceptable carrier or excipient, in an amount effective to treat AMD.

In another embodiment, the present invention provides a method of preventing AMD, by administering to a subject in need thereof a composition comprising at least one agent selected from DIM and I3C, the composition further comprising lycopene, lutein, zeaxanthin, phytoene, phytofluene, carnosic acid or a derivative thereof; and a pharmaceutically acceptable carrier or excipient, in an amount effective to prevent AMD, preferably wet AMD.

In another embodiment, the present invention provides a method of attenuating the progression of AMD, by administering to a subject in need thereof a composition comprising at least one agent selected from DIM and I3C, the composition further comprising lycopene, lutein, zeaxanthin, phytoene, phytofluene, carnosic acid or a derivative thereof; and a pharmaceutically acceptable carrier or excipient, in an amount effective to attenuate the progression of AMD, preferably wet AMD.

In another embodiment, the present invention provides a method of inhibiting the progression of I3C, by administering to a subject in need thereof a composition comprising at least one agent selected from DIM and I3C, the composition further comprising lycopene, lutein, zeaxanthin, phytoene, phytofluene, carnosic acid or a derivative thereof; and a pharmaceutically acceptable carrier or excipient, in an amount effective to inhibit the progression of AMD, preferably wet AMD.

In another embodiment, the present invention provides a method of treating AMD, by administering to a subject in need thereof a composition comprising at least one agent selected from DIM and I3C, the composition further comprising lycopene, lutein, zeaxanthin, phytoene, phytofluene, carnosic acid or a derivative thereof, and a pharmaceutically acceptable carrier or excipient, in an amount effective to treat AMD.

In another embodiment, the present invention provides a method of preventing AMD, by administering to a subject in need thereof a composition comprising at least one agent selected from DIM and I3C, the composition further comprising lycopene, lutein, zeaxanthin, phytoene, phytofluene, carnosic acid, vitamin A, vitamin C, vitamin E, zinc and copper; and a pharmaceutically acceptable carrier or excipient, in an amount effective to prevent AMD, preferably wet AMD.

In another embodiment, the present invention provides a method of attenuating the progression of AMD, by administering to a subject in need thereof a composition comprising at least one agent selected from DIM and I3C, the composition further comprising lycopene, lutein, zeaxanthin, phytoene, phytofluene, carnosic acid, vitamin A, vitamin C, vitamin E, zinc and copper; and a pharmaceutically acceptable carrier or excipient, in an amount effective to attenuate the progression of AMD, preferably wet AMD.

In another embodiment, the present invention provides a method of inhibiting the progression of I3C, by administering to a subject in need thereof a composition comprising at least one agent selected from DIM and I3C, the composition further comprising lycopene, lutein, zeaxanthin,, phytoene, phytofluene, carnosic acid, vitamin A, vitamin C, vitamin E, zinc and copper; and a pharmaceutically acceptable carrier or excipient, in an amount effective to inhibit the progression of AMD, preferably wet AMD.

In another embodiment, the present invention provides a method of treating AMD, by administering to a subject in need thereof a composition comprising at least one agent selected from DIM and I3C, the composition further comprising lycopene, lutein, zeaxanthin, phytoene, phytofluene, carnosic acid, vitamin A, vitamin C, vitamin E, zinc and copper; and a pharmaceutically acceptable carrier or excipient, in an amount effective to treat AMD.

In another aspect, the present invention relates to the use of DIM, in the manufacture of a medicament for preventing, attenuating, inhibiting the progression of or treating AMD, preferably wet AMD.

In another aspect, the present invention relates to the use of I3C, in the manufacture of a medicament for preventing, attenuating, inhibiting the progression of or treating AMD, preferably wet AMD.

As used herein, the term “attenuating” means to soothe, calm or ease the symptoms of any of the aforementioned eye diseases. As used herein, the term “treating” includes preventative as well as disorder remititive treatment.

As used herein, the term “administering” refers to bringing a subject in contact with the formulation of the present invention. In one embodiment, the present invention encompasses administering the formulations of the present invention to a human subject.

In one embodiment, the AMD is wet AMD. In another embodiment, the composition is provided in an amount effective to prevent, inhibit or attenuate the progression of dry AMD to wet AMD.

In one embodiment, the composition comprises DIM. In another embodiment, the composition comprises I3C. In a further embodiment, the composition comprises a combination of DIM and I3C. The active ingredient or ingredients are provided in a collective amount effective to prevent, attenuate, inhibit the progression of, or treating AMD especially wet AMD. For example, for DIM, the composition preferably comprises from about 25 mg to about 500 mg DIM, more preferably from about 50 mg to about 150 mg DIM. For I3C, the composition preferably comprises from about 25 mg to about 500 mg DIM, more preferably from about 50 mg to about 150 mg I3C.

### Pharmaceutical Compositions

Although the active agents DIM and/or I3C, optionally in combinations with carotenoids, adjuvants and/or supplements, can be administered alone, it is contemplated that these compounds will be administered in a pharmaceutical composition containing the active ingredients together with a pharmaceutically acceptable carrier or excipient.

Thus, a further embodiment of the instant invention is pharmaceutical composition in unit dosage form, useful for preventing, attenuating, inhibiting the progression of, or treating AMD, preferably wet AMD, the compositions comprising at least one agent selected from DIM and I3C; and a pharmaceutically acceptable carrier or excipient. Optionally, the composition further comprises at least one carotenoid such as lycopene, lutein and zeaxanthin, at least one adjuvant such as phytoene, phytofluene and carnosic acid or derivatives thereof. The compositions can also comprise minerals such as zinc and copper, and/or vitamins such as vitamin A (e.g., beta carotene), vitamin C and vitamin E.

DIM, I3C and additional agent(s), if provided, can be administered separately or together. It is understood when the compounds are administered separately, administration can be simultaneous or sequential, in any order. For example, a carotenoid can be administered followed by DIM and/or I3C. Alternatively, DIM and/or I3C can be administered prior to the carotenoid. Alternatively, the different components of the combination can be administered together, but in separate dosage forms. Alternatively, the different compounds can be administered together in the same pharmaceutical composition. Further, if more than one of the aforementioned classes of compounds is administered, each component can be administered together or apart from the other. For example if two or more carotenoids are administered, they can be administered in separate dosage forms, simultaneously or sequentially, in any order, or they can be provided in the same pharmaceutical composition, for concurrent administration.

Pharmaceutical compositions for use in accordance with the present invention can be formulated in conventional manner using one or more physiologically acceptable carriers comprising excipients and auxiliaries, which facilitate processing of the active compounds into preparations which can be used pharmaceutically. The active agents are formulated as pharmaceutical compositions and administered to a mammalian subject, such as a human patient in a variety of forms such as liquid, solid, and semisolid. The pharmaceutical compositions can be administered to a subject by any method known to a person skilled in the art, such as orally, topically, parenterally, paracancerally, transmucosally, transdermally, intramuscularly, intravenously, intradermally, subcutaneously, intraperitonealy, intraventricularly, intracranially or intratumorally.

For oral administration, the compounds can be formulated by combining the active compounds with pharmaceutically acceptable carriers known in the art. The compositions can be formulated in any solid or liquid dosage form known in the art, including but not limited to, tablet, caplet, capsule, microcapsule, pellet, pill, powder, syrup, gel, slurry, granule, suspension, dispersion, emulsion, liquid, solution, dragee, bead and beadlet. The oral compositions can be formulated as immediate release formulations, or as controlled or sustained release formulations allowing for extended release of the active ingredient(s) over a predetermined time period. Also, any known means of solubility enhancers for DIM and/or I3C can be used,, including but not limited to nanosizing, inclusion complexes with cyclodextrins, lyposomes, micro/nano emulsions, and the like. Other suitable formulations are those disclosed in U.S. Pat. No. 6,086,915 to Zeligs et al., the contents of which are incorporated by reference herein, are also contemplated.

Suitable excipients for solid formulations include but are not limited to fillers such as sugars, including lactose, sucrose, mannitol, or sorbitol; starch based excipients such as maize starch, wheat starch, rice starch, potato starch and the like, gelatin, gum tragacanth, cellulose based excipients as microcrystalline cellulose, carboxymethylcellulose, hydroxymethylcellulose, hydroxyethylcellulose, methylhydroxypropylcellulose, hydroxypropylcellulose and the like. Polymers such as polyvinylpyrrolidone (PVP) and cross-lined PVP can also be used. In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), surfactants (e.g. sodium lauryl sulfate), and lubricants (e.g. stearic acid, magnesium stearate, polyethylene glycol, sodium lauryl sulfate).

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters. Aqueous carriers include water, alcoholic/aqueous solutions, emulsions or suspensions, including saline and buffered media. Examples of oils include but are not limited to petroleum, animal, vegetable, or synthetic origin, for example, peanut oil, soybean oil, mineral oil, olive oil, sunflower oil, and fish-liver oil.

Preferred oral pharmaceutical compositions include capsules made of gelatin as well as soft, sealed capsules made of gelatin and a plasticizer, such as glycerol or sorbitol. In soft capsules, the active compounds may be dissolved or suspended in suitable liquids, such as fatty oils, liquid paraffin, or liquid polyethylene glycols. In addition, stabilizers may be added. In certain preferred embodiments the capsules exclude components of animal origin and are acceptable for vegetarians and vegans.

Soft gelatin capsules and methods of preparing them are known in the art. Non-limiting examples can be found in U.S. Pat. Nos. 6,217,902; 6,258,380; 5,916,591, and 4,891,229, all of which are incorporated herein by reference.

In another embodiment, the present invention further relates to a composition formulated for application to the eye. The composition can be formulated as drops, solution (aqueous and non-aqueous), cream, paste, ointment, gel, emulsions, suspension, and the like For ophthalmic administration, the composition can be formulated readily by combining the active compounds with pharmaceutically acceptable carriers well known in the art. Such carriers enable the compounds of the invention to be formulated as liquids, gels, emulsions, thermogels, slurries, suspensions, and the like, for ophthalmic use by a patient. Alternatively the composition can be formulated as a solid, for resuspension. In other embodiments, pharmaceutical compositions for ophthalmic administration include aqueous solutions of the active ingredients in water-soluble form.

Other acceptable excipients and additives known to the person with skill in the art may be included in the compositions of the present invention, for example stabilizers, solubilizers, tonicity enhancing agents, buffer substances, preservatives, thickeners, complexing agents and other excipients, as well as additional therapeutic agents.

A solubilizer can be for example, tyloxapol, fatty acid glycerol polyethylene glycol esters, fatty acid polyethylene glycol esters, polyethylene glycols, glycerol ethers or mixtures of those compounds. A specific example of a solubilizer well tolerated by the eye is a polyoxyethylated castor oil for example, the commercial products Cremophor® or Cremophor® RH40. Another example of a solubilizer is tyloxapol. The concentration used depends especially on the concentration of the active ingredient. The amount added is typically sufficient to solubilize the active ingredient. For example, the concentration of the solubilizer is from 0.1 to 5000 times the concentration of the active ingredient.

Examples of buffer substances are acetate, ascorbate, borate, hydrogen carbonate/carbonate, citrate, gluconate, lactate, phosphate, propionate and TRIS (tromethamine) buffers. The amount of buffer substance added is, for example, that necessary to ensure and maintain a physiologically tolerable pH range. The pH range is typically in the range of from 5 to 9, preferably from 5.2 to 8.5.

Tonicity enhancing agents are selected from ionic and non-ionic agents. For example, ionic compounds, include alkali metal or alkaline earth metal halides, such as, for example, CaCl₂ KBr, KCl, LiCl, Nal, NaBr or NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol.

Examples of preservatives are quaternary ammonium salts such as benzalkonium chloride, benzoxonium chloride or polymeric quaternary ammonium salts, alkyl-mercury salts of thiosalicylic acid, such as, for example, thiomersal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid. Where appropriate, a sufficient amount of preservative is added to the ophthalmic composition to ensure protection against secondary contaminations during use caused by microbes.

The compositions of the present invention may comprise further non-toxic excipients, such as, for example, emulsifiers, wetting agents or fillers, such as, for example, the polyethylene glycols (PEG200, 300, 400 and 600) or Carbowax® (Carbowax 1000, 1500, 4000, 6000 and 10000). Other excipients that may be used if desired are listed below but they are not intended to limit in any way the scope of the possible excipients. They can be complexing agents, such as disodium-EDTA or EDTA, antioxidants, such as ascorbic acid, acetylcysteine, cysteine, sodium hydrogen sulfite, butyl-hydroxyanisole, butyl-hydroxytoluene; stabilizers, such thiourea, thiosorbitol, sodium dioctyl sulfosuccinate or monothioglycerol; or other excipients, such as, for example, lauric acid sorbitol ester, triethanol amine oleate or palmitic acid ester.

Optionally, the suspension may also contain suitable stabilizers or agents, which increase the solubility of the compounds, to allow for the preparation of concentrated solutions. For example, U.S. Pat. No. 5,576,311, the contents of which are incorporated by reference herein, teaches stable aqueous suspension of drugs suitable for therapeutic administration to the eye comprising cyclodextrin type-suspending agents. Other useful formulations include submicron ocular emulsions, for example an ocular drug delivery vehicle as disclosed in U.S. Pat. No. 5,496,811, the contents of which are incorporated by reference as if fully set forth herein.

The amount and type of excipient added is in accordance with the particular requirements and is generally in the range of from approximately 0.0001 to approximately 90% by weight.

The amount of a composition to be administered will, of course, depend on many factors including the subject being treated, the severity of the affliction, the manner of administration, and the judgment of the prescribing physician. However, the dose employed will generally depend on a number of factors, including the age and sex of the patient, and the severity of the disease being treated.

Preferably, the preparations are in unit dosage form, intended for oral administration. In such form, the preparation is subdivided into unit doses containing appropriate quantities of the active components. The unit dosage form can be a packaged preparation, the package containing discrete quantities of preparation, for example, tablets, capsules, and powders in vials or ampoules. The unit dosage form can also be a capsule, cachet, or tablet itself or it can be the appropriate number of any of these in packaged form.

The dosing schedule of the compositions of the present invention can vary according to the particular application and the potency of the active ingredients. Determination of the proper dosage is within the skill of the art. For convenience, a single daily dose is preferred. Alternatively, the total daily dosage may be divided and administered in portions during the day such as twice daily, thrice daily and the like. Biweekly, weekly, bimonthly and monthly administration are also contemplated

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Example 1

### Oral Compositions

Preparations were prepared, as follows:

### Preparation 1:

about 25-500 mg DIM (about 10-50% w/w)

about 6 mg lycopene (about 1.5% w/w);

about 8 mg lutein and zeaxanthin (combined—about 2% w/w);

about 0.6 mg of phytoene and phytofluene (combined—about 0.15%); and

about 4 mg carnosic acid (about 1% w/w).

### Preparation 2:

Preparation 2 contains all of the ingredients of preparation 1 and in addition:

about 3 mg β-carotene (about 0.75%);

about 30 mg zinc (about 7.5% w/w);

about 1 mg copper (about 0.25% w/w);

about 50 mg vitamin E (about 12.5% w/w); and

about 50 mg vitamin C (about 12.5% w/w).

### Preparation 3:

Preparation 3 comprises the following ingredients

| | | | | LM¹ | Lut | β-car | LR² | | | Tocopherol | Ascorbic |
|---|---|---|---|---|---|---|---|---|---|---|---|
| | Ingredient | mg (%) | | 6% G | 20% G | 30% G | 40% | ZnO | CuO | Acetate | acid |
| 1 | DIM | 100 | (ca. 40) | | | | | | | | |
| 2 | Lycopene | 6 | (1.5) | 100 | | | | | | | |
| 3 | Lutein/zea | 8 | (2) | | 40 | | | | | | |
| 4 | β-carotene | 3 | (0.75) | | | 10 | | | | | |
| 5 | Carnosic acid | 4 | (1) | | | | 10 | | | | |
| 6 | Phy + Phf | 0.6 | (0.15) | 100 | | | | | | | |
| 7 | Vitamin E | 50 | (12.5) | | | | | | | 50 | |
| 8 | Vitamin C | 50 | (12.5) | | | | | | | | 50 |
| 9 | Zinc | 30 | (7-5) | | | | | 37.5 | | | |
| 10 | Copper | 1 | (0.25) | | | | | | 1.25 | | |

| | |
|---|---|
| Total (mg) | 398.75 |

¹Lyc-O-Mato ®
²Carnosic Acid

The above compositions are mixed with mineral oil and or vegetable oil and are prepared as soft gelatin capsules containing about 400 mg total weight.

Alternatively the above compositions are admixed with fillers and excipients and are prepared as solid oral dosage forms containing about 400 mg total weight.

The same formulations were prepared, containing I3C in replacement of DIM.

### Example 2

### Ophthalmic Compositions

Each of preparations 1 to 3 above were mixed with an ophthalmically acceptable carrier and formulated into ophthalmic formulations.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

What is claimed is:

1. A method of preventing, attenuating, inhibiting the progression of, or treating age-related macular degeneration (AMD), comprising administering to a subject in need thereof a composition comprising at least one agent selected from 3,3′-diindolylmethane (DIM) and indole-3-carbinol (I3C); and a pharmaceutically acceptable carrier or excipient, in an amount effective to prevent, attenuate, inhibit the progression of, or treat AMD.

2. The method according to claim 1, wherein the AMD is wet AMD.

3. The method according to claim 1, wherein the composition is effective at preventing, attenuating or inhibiting the progression of dry AMD to wet AMD.

4. The method according to claim 1, wherein the composition comprises DIM.

5. The method according to claim 4, wherein the composition comprises from about 25 mg to about 500 mg DIM.

6. The method according to claim 5, wherein the composition comprises from about 50 mg to about 150 mg DIM.

7. The method according to claim 1, wherein the composition comprises I3C.

8. The method according to claim 1, wherein the composition comprises DIM and I3C.

9. The method according to claim 1, wherein the composition further comprises at least one carotenoid.

10. The method according to claim 9, wherein the carotenoid is lycopene.

11. The method according to claim 10, wherein the lycopene is provided as a natural tomato extract.

12. The method according to claim 11, wherein the lycopene is extracted from tomato oleoresin.

13. The method according to claim 1, wherein the composition further comprises phytoene, phytofluene, lutein, zeaxanthin and carnosic acid or a derivative thereof.

14. The method according to claim 1, wherein the composition further comprises at least one mineral.

15. The method according to claim 14, wherein the at least one mineral is zinc.

16. The method according to claim 15, wherein the zinc is provided as zinc oxide.

17. The method according to claim 14, wherein the at least one mineral is copper.

18. The method according to claim 17, wherein the copper is provided as cupric oxide.

19. The method according to claim 1, wherein the composition further comprises at least one vitamin.

20. The method according to claim 19, wherein the at least one vitamin is selected from the group consisting of vitamin A, vitamin C, vitamin E, derivatives thereof and combinations thereof.

21. The method according to claim 1, wherein the composition comprises DIM, lycopene, phytoene, phytofluene, lutein, zeaxanthin, and carnosic acid or a derivative thereof.

22. The method according to claim 21, wherein the composition further comprises vitamin A, vitamin C, vitamin E, zinc and copper.

23. The method according to claim 1, wherein the composition further comprises tomato oleoresin.

24. The method according to claim 1, wherein the composition is administered orally.

25. The method according to claim 24, wherein the composition is in a form selected from the group consisting of a tablet, caplet, capsule, microcapsule, pellet, pill, powder, syrup, gel, slurry, granule, suspension, dispersion, emulsion, liquid, solution, dragee, bead and beadlet.

26. The method according to claim 25, wherein the capsule is selected from a soft gelatin capsule and a hard gelatin capsule.

27. The method according to claim 1, wherein the composition is administered topically to the eye.

28. The method according to claim 27, wherein the composition is in a form selected from the group consisting of a solution, eye drops, liquid, cream, paste, ointment, emulsion, submicron emulsion, gel, thermogel, semi-solid, solid and suspension.

29. The method according to claim 1, wherein the composition further comprises at least one solubility enhancer to enhance the solubility of DIM, I3C or the combination thereof.

30. A pharmaceutical composition comprising at least one agent selected from 3,3′-diindolylmethane (DIM) and indole-3-carbinol (I3C), the composition further comprising lycopene, lutein, zeaxanthin, phytoene, phytofluene, carnosic acid or a derivative thereof, and a pharmaceutically acceptable carrier or excipient.

31. The composition according to claim 30, wherein the composition comprises DIM.

32. The composition according to claim 31, comprising from about 25 mg to about 500 mg DIM.

33. The composition according to claim 32, comprising from about 50 mg to about 150 mg DIM.

34. The composition according to claim 30, wherein the composition comprises I3C.

35. The composition according to claim 30, wherein the composition comprises DIM and I3C.

36. The composition according to claim 30, wherein the lycopene is provided as a natural tomato extract.

37. The composition according to claim 36, wherein the lycopene is extracted from tomato oleoresin.

38. The composition according to claim 30, further comprising at least one mineral.

39. The composition according to claim 38, wherein the at least one mineral is zinc.

40. The composition according to claim 39, wherein the zinc is provided as zinc oxide.

41. The composition according to claim 38, wherein the at least one mineral is copper.

42. The composition according to claim 39, wherein the copper is provided as cupric oxide.

43. The composition according to claim 30, further comprising at least one vitamin.

44. The composition according to claim 43, wherein the at least one vitamin is selected from the group consisting of vitamin A, vitamin C, vitamin E, derivatives thereof and combinations thereof.

45. The composition according to claim 30, wherein the composition further comprises vitamin A, vitamin C, vitamin E, zinc and copper.

46. The composition according to claim 30, wherein the composition further comprises tomato oleoresin.

47. The composition according to claim 30, wherein the composition is formulated for oral administration.

48. The composition according to claim 47, in a form selected from the group consisting of a tablet, caplet, capsule, microcapsule, pellet, pill, powder, syrup, gel, slurry, granule, suspension, dispersion, emulsion, liquid, solution, dragee, bead and beadlet.

49. The composition according to claim 48, wherein the capsule is selected from a soft gelatin capsule and a hard gelatin capsule.

50. The composition according to claim 30, wherein the composition is formulated for topical administration to the eye.

51. The composition according to claim 50, in a form selected from the group consisting of a solution, eye drops, liquid, cream, paste, ointment, emulsion, submicron emulsion, gel, thermogel, semi-solid, solid and suspension.

52. The composition according to claim 30, further comprising at least one solubility enhancer to enhance the solubility of DIM, I3C or the combination thereof.

53. A pharmaceutical composition comprising 3,3′-diindolylmethane (DIM), lycopene, lutein, zeaxanthin, phytoene, phytofluene, carnosic acid or a derivative thereof; and a pharmaceutically acceptable carrier or excipient.

54. A pharmaceutical composition comprising 3,3′-diindolylmethane (DIM), lycopene, lutein, zeaxanthin, phytoene, phytofluene, carnosic acid or a derivative thereof, vitamin A, vitamin C, vitamin E, zinc, copper; and a pharmaceutically acceptable carrier or excipient.

55. Use of 3,3′-diindolylmethane (DIM) in the manufacture of a medicament for preventing, attenuating, inhibiting the progression of, or treating age-related macular degeneration (AMD).

56. Use according to claim 55, wherein the AMD is wet AMD.

57. Use of indole-3-carbinol (I3C) in the manufacture of a medicament for preventing, attenuating, inhibiting the progression of, or treating age-related macular degeneration (AMD).

58. Use according to claim 57, wherein the AMD is wet AMD.
